(19) United States
(12) Patent Application Publication
(10) Pub. No.: US 2012/0172326 A1
(43) Pub. Date: Jul. 5, 2012

(54) Moisturizing Mixture, Cosmetic And/Or Pharmaceutical Compositions Containing The Moisturizing Mixture, Use Of The Moisturizing Mixture, And Cosmetic Method

(75) Inventors: Patricia Da Luz Moreira, Sao Paulo SP (BR); Adriano Tadeu Siqueira Jorge, Guarulhos - SP (BR); Juliana Palmeira Portugal Marques, Agua Santa SP (BR); Jean Luc Gesztesi, Sao Paulo - SP (BR); Philip Leite Ribeiro, Sao Paulo - SP (BR); Sara Rocha, Osasco - SP (BR)

(73) Assignee: Natura Cosmeticos S.A., Itapecerica da Serra (BR)

(21) Appl. No.: 13/131,962

(22) PCT Filed: Nov. 27, 2009
(86) PCT No.: PCT/BR2009/000390

§ 371 Date: Feb. 28, 2012

(30) Foreign Application Priority Data
Nov. 28, 2008 (FR) 0858116

(51) Int. Cl. A61K 31/715; A61K 31/23; A61K 31/231; A61K 31/232; A61P 17/00; A61K 31/197; A61K 8/92; A61K 8/44; A61K 8/73; A61Q 19/00; A61K 31/164; A61K 31/201

(52) U.S. Cl. 514/54; 514/625; 514/547; 514/559; 514/561; 514/564

(57) ABSTRACT

The present invention refers to a moisturizing mixture for sensitive skins, atopic dermatitis and/or Ichthyosis vulgaris containing a synergistic combination among its constituents, and cosmetic and/ pharmaceutical compositions containing said moisturizing mixture. The invention also refers to a moisturizing mixture comprising a synergistic combination between at least one cutaneous-barrier repairing agent; NMF-precursor amino acids; and at least one filagrin-synthesis stimulating agent. Cosmetic and/or pharmaceutical compositions comprising said moisturizing mixture and a physiologically acceptable carrier are also described in the present invention.

The present invention relates to a moisturizing mixture for sensitive skins, atopic dermatitis and/or Ichthyosis vulgaris, containing a synergistic combination among its constituents, and cosmetic and/or pharmaceutical compositions containing said moisturizing mixture.

### BACKGROUND OF THE INVENTION

In the cosmetic and dermatological field, compositions that exhibit combinations of moisturizing ingredients, claiming a skin-moisturizing potential, are well known.

Brazilian patent application PI 0408373-3 discloses compositions for treating skin, which repair and increase the function of the cutaneous barrier. These compositions contain, in combination, glycerin, vitamin B3 and amino acids. As a result, they promote moisturizing of the skin, and, further, vitamin B3 acts like a repair element of the cutaneous barrier.

United States document U.S. Pat. No. 6,906,106 describes a composition for topical application to the skin, which comprises fibers of specific length and at least one ubiquinone. In addition, said document teaches that one of the options of ubiquinones that can be employed in said composition is coenzyme Q10. Said composition further comprises an active ingredient that, in turn, may be selected from a specified group, wherein the moisturizing agents, ceramides, vitamins and antioxidants are included.

United States document US 2007/0105996 describes a composition containing: an aqueous phase dispersed in an oil phase; at least one component of a specified formula with hydroxyalkyl radicals and its salts and solvates; and at least one polyolefinic emulsifier with a polar part. The objective is to provide a hydroxyalkylurea component with improved moisturizing properties and a cosmetic composition for application on keratinic matters, for care or make-up thereof.

Another United States document, U.S. Pat. No. 6,136,851, discloses esters of a specified formula, such as: alpha-tocoferol, beta-tocoferol, gama-tocoferol esters, among others.

Further, a cosmetic and/or pharmaceutical composition is also defined in said document, which comprises an ester, according to the above definition, as an active ingredient of the preparation and a physiologically acceptable carrier. Said composition comprises an oil phase composed by at least one oil selected from a specific group, wherein triglycerides, jojoba oil, among others, are listed.

As it can be noted, the state of the art documents do not disclose moisturizing mixtures essentially intended for sensitive skins, which contain synergistic combinations among their constituents, so that, when applied to the skin of the user, they can promote prolonged moisturizing of this organ, accompanied by benefits of repair of the cutaneous barrier.

### BRIEF DESCRIPTION OF THE INVENTION

An objective of the present invention is to provide a moisturizing mixture intended for sensitive skins, which exhibits a synergistic combination among its components. This moisturizing mixture comprises a synergistic combination among:

a) at least one agent for the repair of cutaneous barrier;
b) at least one NMF-precursor amino acid; and
c) at least one stimulating agent of the synthesis of filagrin.

It is also an objective of this invention to provide cosmetic and/or pharmaceutical bases, in which the moisturizing mixture is applied. This cosmetic and/or pharmaceutical composition comprises:

a) a synergistic moisturizing mixture containing at least one agent that repairs the cutaneous barrier; at least one NMF-precursor amino acid; and at least one agent that stimulates the synthesis of filagrin; and
b) a physiologically acceptable carrier.

### BRIEF DESCRIPTION OF THE DRAWINGS

The present invention is better described in greater details with reference to the examples represented in the drawings. The figures show:

FIG. 1—a schematic cross-sectional view of the epidermis structure with its various layers: (1) stratum corneum; (2) extracellular lipids; and (3) corneocytes;

FIG. 2—a structure of the cornified envelope under the plasmatic membrane;

FIG. 3—a molecular structure of a ceramide;

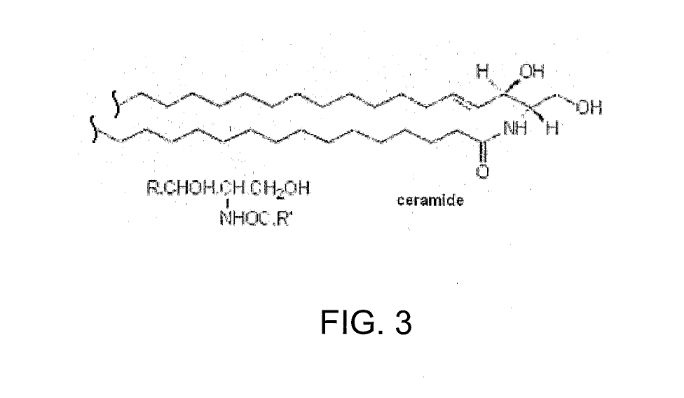

FIG. 4—a graph of results obtained in the complete-corneometry test in use;

FIG. 5—a graph of non-linear regression curves obtained after 7 days of TEWL test as a function of the number of “tape-stripping” removals; and

FIG. 6—a graph of non-linear regression curves obtained after 15 days of TEWL test as a function of the number of “tape-stripping” removals.

### DETAILED DESCRIPTION OF THE INVENTION

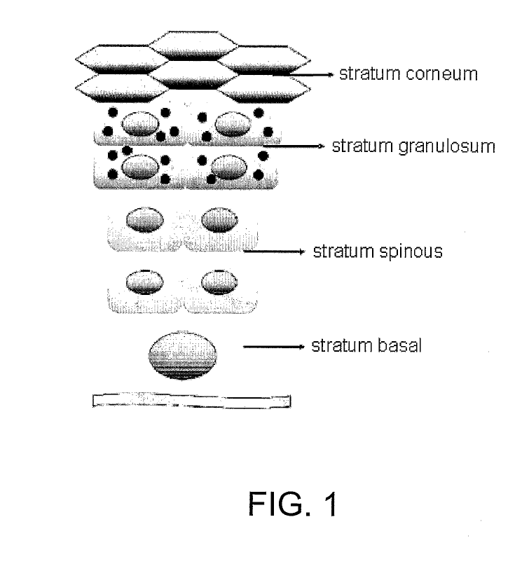

The skin is composed of different several morphologically different layers, the outermost one being known as stratum corneum (cited hereinafter as SC). This superficial region, only 10-20 micrometers deep, is the primary barrier for cutaneous absorption of compounds, as well as for loss of water. Below the stratum corneum lies the viable epidermis with 50-100 micrometers of thickness, which is responsible for generating the SC. The viable epidermis is a stratified scaly epithelium, formed by basal, spinous and granulous layers, as illustrated in FIG. 1. Each layer is defined by the position, shape, morphology and differentiation status of the keratinocytes. The epidermis is a dynamic tissue that renews itself constantly, in which the loss of cells from the SC surface through the peeling off process is balanced by the cell growth in the lower epidermis (basal layer).

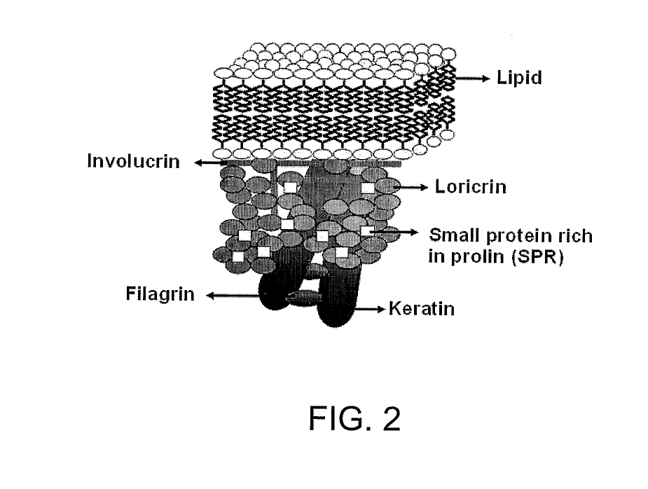

After leaving the basal layer, the keratinocytes begin to differentiate and, during their apical migration through the spinous and granulous strata, they undergo a number of changes in both the structure and their composition, wherein they synthesize and express numerous and different structural proteins and lipids during their maturation. The final steps of differentiation of the keratinocytes are associated with deep changes in their structure, resulting in their transformation into corneocytes. The corneocytes are scaly, non-nucleated, flattened cells, concentrated in karatin filaments and enclosed in a cellular envelope composed of cross-linked proteins, as well as a lipidic outer envelope linked covalently, as illustrated in FIG. 2 (Candi et al., 2005). This extracellular lipidic envelope forms a hydrophobic matrix that also acts maintaining osmotically active materials inside the corneocytes. At the end of the differentiation process, characteristic organelles (lamellar bodies) appear in the cells of the granular layer. The lamellar bodies, which are essential in forming the SC, are ovoid organelles enriched mainly with polar lipids and enzymes, which are necessary for the formation of the SC. In reply to certain signals (including the increase in the concentration of calcium), the lamellar bodies move to the surface and periphery of the cells of the granular layer and merge with the plasmatic membrane to secrete their contents in the extracellular space by exocytosis. The lipids derived from the lamellar bodies are subsequently modified and rearranged in an intracellular lamella oriented approximately parallel to the cell surface.

The lamellar bodies act as carriers of the precursors of the SC barrier lipids, constituted mainly by glycosphingolipids, free sterols and phospholipids. After release of the lipids in the interface between stratum granulosum and SC, the polar lipid precursors are enzymatically converted into non-polar products arranged in the outer lamellar structures. The glycolipid hydrolysis generates ceramides, while the phospholipids are converted into free fatty acids.

The main formation mechanisms and the composition of the cutaneous barrier and viable epidermis are described below.

Lipidic Composition of the SC (Stratum Corneum):

The majority lipidic components of the stratum corneum are ceramides, cholesterol and free fatty acids, constituting approximately 50%, 25% and 10% of the total mass of lipids, respectively. However, this proportion may change in the lower layers of the skin. Ceramides consist of long chains or sphingoid bases (aliphatic chains of amines containing 2 or 3 hydroxyl groups and a trans-type double bond in position 4, attached to a fatty acid via an amide bond).

The ceramide head groups (CER) are very small and contain several functional groups that can form side hydrogen bridges with adjacent CER molecules. The size distribution of the aliphatic chain of CER is bimodal, like most of the chains with size between C24-C26. Only a small portion of CER has aliphatic chains of size C16-C18. The chain size of C24 and C26 is much longer than that one of the phospholipids in plasmatic membranes. Nine subclasses of ceramides (CER) were identified in the human stratum corneum. They differ from each other by the architecture of the head group (sphingosine, phytosphyngosine or 6-hydroxysphingosine) bonded to a fatty acid or a alpha-hydroxy fatty acid, with a variable chain size.

Ceramides of types 1, 2 and 5 are based on sphingosine. On the other hand, ceramides of types 3, 4, 6, 7, 8 and 9 are formed from phytosphingosine.

Choleterol is the main sterol of the human SC. As the phospholipids are degraded in the final phases of the differentiation process, the remaining unsaturated fatty acids, particularly oleic acid, are transferred to the cholesterol in order to produce cholesterol esters. The fraction of free fatty acids consists mainly of saturated acids with chain sizes of C22 and C24, but they may present a size ranging from C14 to C28. This fraction is the only ionizable in the human SC and, therefore, it is essential in forming the lamellae.

Cornified Envelope of the SC and NMF (Natural Moisturizing Factor):

The cornified envelope consists of a complex mixture of covalently cross-linked proteins, coupled to the plasmatic membrane of the cells. On the cytoplasmatic side, keratin filaments are enclosed by the cornified envelope.

The components of the envelope are cross-linked mainly by bonds via lysine ε-(γ-glutamyl) isopeptide, catalyzed by calcium-dependent transglutaminases (Candi et al., 2005). These bonds lead to a protein complex that is highly insoluble and resistant to the conventional biochemical extraction procedures.

Various protein components of the envelop were identified: involucrins, a small protein rich in prolin (SPR), loricrin, cystalin, desmoplaquin, envoplaquin, periplaquin, elafin, filagrin, proteins S100, repetins and several keratins.

Prophylagrin is expressed in the granular layer of the epidermis and rapidly processed while the queratinocytes continue the terminal differentiation process for the cornified layer. The processing of prohilagrin involves the action of two or more phosphatases that dephosphorylize prophilagrin, which is specifically proteolyzed by endoproteinases to generate philagrin and the N-terminal peptide.

The N-terminal peptide accumulates in the nucleus of the cells of the granular and transition layers and is co-localized in the condensed chromatin of the transition cells that are in the nuclear breakup process (apoptosis). Due to this fact, it is believed that the N-terminal peptide should be directly or indirectly related to the apoptosis process of the queratinocytes of the granular layer into corneocytes.

In the upper granular layers, filagrin is degraded into free amino acids, forming the component known as natural moisturizing factor (NMF). The NMF is important to maintain the flexibility and osmolarity of the skin. The degradation process begins after the keratins (particularly K1) and filagrin undergoing deamination by the enzyme peptidyl arginine deaminase (PAD), which converts the arginine peptidic binding to citrulin. This chemical modification alters the load of both filagrin and keratin, causing these proteins to dissociate. Filagrin is subsequently hydrolyzed to free amino acids by proteinases such as catepsin L and B in the acidic medium of the SC.

A small amount of filagrin escapes from the complete proteolysis and is incorporated into the cellular envelope, where it is usually found cross-linked with loricrin.

Ichthyosis vulgaris (IV) is a genetic disorder of the skin, which occurs worldwide and affects all the races (Irvine and Paller, 2002). Clinically, it appears in the first months/years of life with the scaling of the skin, being generally more marked at the ends, indicating a deficient cutaneous barrier. Approximately 8% of the patients who suffer from atopic dermatitis also present clinical signs of IV (Tay et al., 1999). Immunohistochemistry studies, ELISA (Seguchi et al., 1996) and microarray (Sugiura et al., 2005) have previously demonstrated a decrease in the level of filagrin, as well as in the level of filagrin RNA expression in skins with atopic dermatitis and certain mutations in its coding gene (Palmer et al., 2006). Recently, it has also been identified mutations in the gene of filagrin as being the cause of the symptoms presented by patients with IV (Smith et al., 2006). Thus, the importance of this protein in the skin, for the latter to perform its protective barrier function properly, becomes evident.

Moisturizing Mixture with a Synergistic Effect of the Present Invention:

The present invention describes a moisturizing mixture particularly indicated for sensitive skin, which comprises a synergistic combination between:

a) at least one agent for the repair of cutaneous barrier;
b) at least one NMF-precursor amino acid; and
c) at least one stimulating agent of the synthesis of filagrin.

This mixture promotes a differentiated and prolonged moisturizing, since it works with different moisturizing mechanisms, exhibiting synergism and acting at cellular level. This mixture combines, in a synergistic manner, two moisturizing strategies: cutaneous barrier repair and cellular mechanisms.

This mixture provides moisturizing by virtue of the:

reinforcement of skin barrier, by both adding lipids exogenously and stimulating the production of lipids by the cells themselves;

addition of amino acids that compose the NMF of the skin;

stimulation of filagrin synthesis;

stimulation of AQ3 synthesis.

The present invention relates to a moisturizing mixture for sensitive skins, atopic dermatitis and/or Ichthyosis vulgaris. For the purposes of the invention, the expression “sensitive skins” refers to all kind of skins that presents any adverse reaction during the use of any cosmetic product available in the market. An “adverse reaction” relates to an allergic process that can be observed by flush/redness, itch, skin roughness and any other altered aspect of the skin concerning, particularly, color and texture.

The components of the moisturizing mixture of the present invention are described in detail below.

### a) Skin-Barrier Repair Agent:

Preferably, the cutaneous-barrier agent is selected from the group:

Exogenous lipids:
i) ceramides and analogs thereof of a space chemical structure such as demonstrated below:

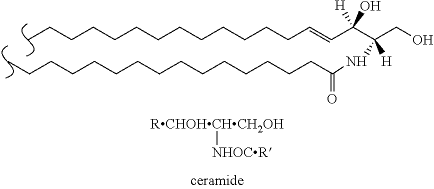

wherein R=R′ refers to C16-30:0:2 and R′=R; and/or
ii) triglycerides (butters and oils) composed either individually and/or in mixture of 2 or more fatty acids belonging to the group: caprylic acid (C8:0); capric acid (C10:0); lauric acid (C12:0); myristic acid (C14:0); palmitic acid (C16:0); palmitoleic acid (C16:1); margaric acid (C17:0); margaroleic acid (C17:1); stearic acid (C18:0); oleic acid (C18:10); linoleic acid (C18:2); linonenic acid (C18:3); arachidic acid (C12:0); gadoleic acid (C20:1); and/or
iii) cyclic fatty acids with a long-chain cyclopentanic ring like chaulmougric acid, hidnocarpic acid and gorlic acid.
Stimulation of the synthesis of lipids: the main example of this class is the product Recoverine, supplied by Silab.
Stimulation of the synthesis of envelope proteins: the main example of this class is the product Recoverine=involucrin, supplied by Silab.

In the preferred embodiments, at least one cutaneous-barrier repair agent is added to the moisturizing mixture of the present invention in an amount ranging from 0.1% to 5.0% by weight, based on the total weight of the cosmetic composition and pharmaceutical composition, and ranging from 1.0% to 49.0% by weight, based on the total weight of the moisturizing mixture.

b) NMF-Precursors Amino Acids:

NMF is a complex mixture of low-molecular-weight moisturizers (Table 1), which include pyrrolidonic carboxylic acid (PCA), a cycled glutamine derivative; urocanic acid, derived from histidine by action of histidase and citrulin, produced from arginine by PAD. These free amino acids derived from filagrin are highly hygroscopic and can absorb a large amount of water, even in an environment having low relative humidity.

Table 1 below lists the components of the NMF:

TABLE 1

| Components of the NMF | % in the composition |
|---|---|
| Free amino acids | 40 |
| Pyrrolidonic carboxylic acid | 12 |
| Lactate (tallow) | 12 |
| Sugars (glycosylceramide) | 8.5 |
| Urea (tallow) | 7 |
| Chloride | 6 |
| Sodium | 5 |
| Potassium | 4 |
| Ammonia, uric acid, glucosamine | 1.5 |
| and creatine | |
| Calcium | 1.5 |
| Magnesium | 1.5 |
| Phosphate | 0.5 |
| Citrate and formiate | 0.5 |

The amino acids that constitute the NMF precursor groups are selected respecting the proportion described below, in Table 2:

TABLE 2

| Item | Composition (%) |
|---|---|
| Acidic amino acids | 11 |
| Hydroxy-amino acids | 31 |
| Neutral amino acids | 36 |
| Aromatic amino acids | 3 |
| Cyclic amino acids | 2 |
| Basic amino acids | 15 |
| Others | 2 |

Said NMF-precursor amino acids are selected from the group of compounds having the following chemical structure:

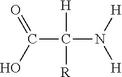

wherein R may be: H; CH₃; COH₃; C₂₀H₄; C₂O₂H₃; C₃O₂H₅; C₃H₅; C₃H₇; C₄H₉; C₄NH₁₀; C₄N₂H₅; C₄N₃H₁₀; C₇H₈; C₇OH₈.

Examples of NMF-precursor amino acids that may be added to the moisturizing mixture of the present inention are Prodew 500+alanine+serine, supplied by Ajinomoto.

In the preferred embodiments, the NMF-precursor amino acids are added to the moisturizing mixture of the present invention in amounts ranging from 0.1% to 10% by weight, based on the total weight of the cosmetic composition and pharmaceutical composition and ranging from 1.0% to 98.0% by weight, based on the total weight of the moisturizing mixture.

c) Filagrin Synthesis Stimulating Agent:

Preferably, the agent that stimulates the synthesis of filagrin is the product Filaggryl®, available from Cognis Corporation.

According to the Material Safety Data Sheet available from Cognis, Filaggryl® refers to a complex preparation of natural origin, of cosmetic use, comprising the following formulation, shown in Table 3:

TABLE 3
FILAGGRYL ®

| Chemical compound | Concentration (%) |
|---|---|
| Manitol (CAS 69-65-8) | >50.0 |
| Hydrolized casein (CAS 65072-006) | 10.0-25.0 |
| ARGININE (CAS 74-79-3) | 10.0-25.0 |
| Serine (CAS 56-45-1) | 5.0.-10.0 |
| Treonine (CAS 72-19-5) | 1.0000-5.00 |
| Hydrolyzed soybean protein (CAS | 1.0000-5.00 |
| 68607-88-5) | |
| Histidine hydrochloride (CAS 1007-42-7) | 1.0000-5.00 |
| Calcium pantotenate (CAS 137-08-6) | 1.0000-5.00 |

In the preferred embodiments, the filagrin synthesis stimulating agent is added to the moisturizing mixture of the present invention in an amount ranging from 0.1% to 5.0% by weight, based on the total weigh of the cosmetic composition and pharmaceutical composition and ranging from 1.0% to 49.0% by weight, based on the total weight of the moisturizing mixture.

Aquaporin:

Optionally, the moisturizing mixture of the present invention may further contain an agent that stimulates the expression of aquaporins.

Aquaporins are families of small proteins (≈30 Kda/monomer), hydrophobic and integral of the membrane, which are expressed in a variety of animals and the vegetable kingdom. In mammals, 13 aquaporins were described so far (Verkaman, 2005), these being expressed in many epithelia and endothelia involving the transportation of fluids, as well as in some types of cells that do not make the transportation of fluids necessarily as the skin, fat and bladder cells do.

Table 4 below details functions and expressions of aqaquaporins.

TABLE 4

| AQ | Permeability | Expressed tissue |
|---|---|---|
| 0 | water | Eyes |
| 1 | water | Kidney, endothelium, |
| | | erythrocytes, intestines, |
| | | cornea |
| 2 | water | Kidney |
| 3 | water, glycerol | Kidney, epidermis, |
| | | conjunctive, respiratory |
| | | tract, bladder |
| 4 | water | Brains, dorsal spine, |
| | | kidney, grand |
| | | epithelium, skeleton |
| | | muscle, stomach, retina |
| 5 | water | Grand epithelium, |
| | | cornea, lung epithelium, |
| | | gastrointestinal tract |
| 6 | Chlorine | kidney |
| 7 | Water, glycerol | Adipose disuse, testi- |
| | | cles, kidney |
| 8 | water | Liver, pancreas, |
| | | intestines, salivary |
| | | gland, testicle, heart, |
| 9 | water | Liver, white blood cells, |
| | | testicle, brains |
| 10 | water | Intestines |
| 11 | | Kidney, liver |
| 12 | | pancreas |

The basal layers of keratinocytes express aquaporin-3 (AQ30) that facilitates the transportation of water from the dermis to the SC.

Glycerol is an important intermediate of the energetic metabolism, a substrate for biosynthesis of several lipids, osmolarity regulator and a chemical chaperone responsible for a number of biological processes, such as glycogenesis, lipid synthesis, glucose homeostase, osmoregulation and apoptosis (Zheng et al., 2003). Glycerol has been used for years in cosmetic formulations as a wetting agent and a moisturizing agent.

The greatest importance of glycerol×transportation of water by AQ3 is understood through a biophysical perspective. AQ3 is expressed in the basal layer of the keratinocytes, where the movement of water from the dermis to the surface layers of the epidermis and the SC is guided by small osmotic gradients, produced by the slow loss of water through evaporation of the surface of the SC. The loss of transepidermal water indicates a flow of water of 10 nl/min/cm2 of skin in basal conditions, which may increase to >100 nl/min/cm2 after removal of SC (Ghadially et al., 1996; Haratake et al., 1997).

These flows are much lower than that >5×1010 nl/min/cm2 in the kidney tubules and exocrine glands, such as salivary glands, where the transportation of water mediated by aquaporin is very important (Schnermann et al., 1998). However, the transportation of glycerol in the epidermis is very slow and, therefore, its transportation rate is sensitive to the intrinsic permeability of glycerol in the basal layer of the keratinocytes (Nara et al., 2003).

Aquaporins Stimulating Agent

A preferred example of an active to stimulate the synthesis of aquaporin-3 is Aquaphyline, supplied by Silab.

The agent that stimulates the synthesis of aquaporin, optionally employed in the synergistic moisturizing mixture of the present invention, is selected from the polysaccharides (CH₂O)n, including urocanic acids, represented by the chemical structures below.

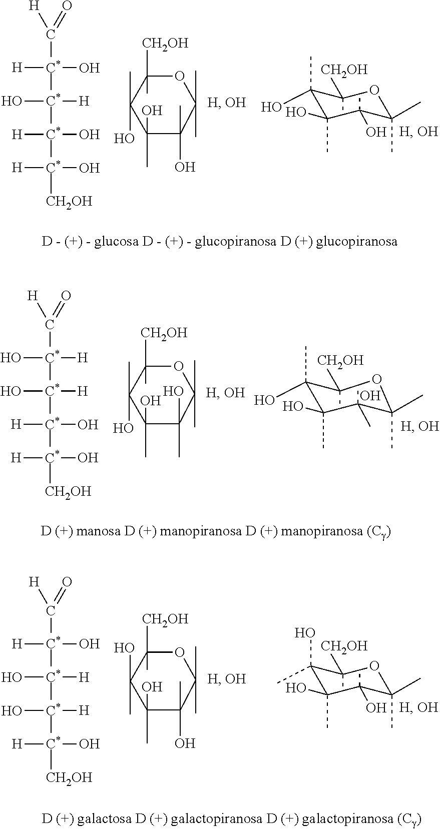

In optional embodiments, at least one aquaporin expression stimulating agent is added to the moisturizing mixture of the present invention in an amount ranging from 0.1% to 5.0% by weight, based on the total weight of the cosmetic composition and pharmaceutical composition and ranging from 1.0% to 49.0% by weight, based on the total composition of the moisturizing mixture.

Cosmetic and Pharmaceutical Compositions

The cosmetic and pharmaceutical compositions comprise from 0.3% to 10.2% of the moisturizing mixture of the present invention, by weight, based on the total weight of said compositions and a carrier.

In preferred embodiments, the cosmetic and pharmaceutical compositions comprise:

a) a synergistic moisturizing mixture containing at least one cutaneous-barrier repair agent; NMF-precursor amino acids; and at least one filagrin-synthesis stimulating agent; and
b) a physiological acceptable carrier.

The cosmetic and pharmaceutical compositions that comprise the moisturizing mixture of the present invention may comprise a few components already known from the state of the art.

The cosmetic and pharmaceutical compositions that comprise the moisturizing mixture of the present invention may further contain several components that have specific functions required for each specific situation.

Some of these compounds, which may be added to said compositions are as follows:

main active substances (either encapsulated or not): may be lipophilic or hydrophilic, like seaweed extracts, combination of palmitoyl hydroxypropyl trimonium aminopectin, glycerin cross-polymer, lecithin and grape-seed extract, bisabolol (anti-inflammatory active), D-pantenol (conditioning active), biosaccahride gum 2 and biosaccharide gum 3 and other actives usually added to compositions of products for topical use;

bacteriostatics, bactericides or antimicrobials;

emulsifiers like potassium cetylphosphate, among others;

emollients;

wetting agents;

stabilizing agents like sodium chloride, among others;

scavenging agents like ethylenediaminetetracetic acid (EDTA) and salts thereof, among others;

pH adjusting agent like trietanolamine, sodium hydroxide, among others;

preserving agents like fenoxyethanol, PEG-5 Ccoate and PEG-8 dicocoate and iodopropinyl butylcarbamate and PEG-4, among others;

dyes of natural or synthetic origin;

thickening agents like xanthan gum and carbomer, among others;

plant extracts: chamomile, rosemary, thyme, calendula, carrot extract, common juniper extract, gentian extract, cucumber extract, among others;

skin conditioning agents; and

other cosmetically acceptable components, which are compatible with the moisturizing mixture of the present invention.

The main examples of products that may be prepared starting from the moisturizing mixture of the present invention or from cosmetic and pharmaceutical compositions comprising said mixture are:

body moisturizing milk;

face moisturizing milk;

body moisturizing lotion;

face moisturizing lotion;

gels;

products for the scalp;

sunscreens or sun blockers for adults and children, either intended for concomitant use with sports practice of not;

body or face moisturizers;

body or face anti-spot products;

body or face firming products;

self-tanning products;

insect repelling products;

skin-brightening body or face moisturizers;

pharmaceutical preparations of topical application;

body and face cosmetic preparations for children;

localized-action cosmetic preparations, specific for periocular regions, lip contours, lips, anti-spot, anti-bag, among others;

antiacne products;

products for enlightening the skin;

pharmaceutical compositions for the treatment of specific dermatoses;

lipsticks and waxy bases;

blushes and pigmented bases;

any make-up product for the eye area;

other cosmetic and pharmaceutical products.

The present invention relates to a moisturizing mixture for sensitive skins, atopic dermatitis and/or Ichthyosis vulgaris. For the purposes of the invention, the expression “sensitive skins” refers to all kind of skins that presents any adverse reaction during the use of any cosmetic product available in the market. An “adverse reaction” relates to an allergic process that can be observed by flush/redness, itch, skin roughness and any other altered aspect of the skin concerning, particularly, color and texture.

Description of the Tests:

Corneometry (Screening) Test:

This test was carried out on 42 women (10 women/product) with age between 18 and 45 years with dry skin characteristic in the formearm region. The moisturizing kinetics used was of 2 hs, 8 hs, 12 hs and 24 hs. The statistic comparison was carried out by ANOVA and Tukey test (p<0.05) between products versus placebo and versus control. The assessments were carried out with Corneomete® 525.

Complete Comeometry Test:

The same protocol detailed above was followed, but 20 women/product were used in this case, totaling 120 women and the evaluations of kinetics were made with 2 hs, 8 hs, and 24 hs at T0 and T15. The products were used twice a day for 15 days.

TEWL Test (Transepidermal Water Loss):

This test evaluates the mount of water that migrates from the dermis to the epidermal layers and consequently to the atmosphere via diffusion and evaporation.

In the TEWL test, 90 women with age between 18 and 45 years with dry skin characteristic in the forearm region were used (10 women/product). The evaluation was carried out after each 10 “tapes stripping”, totaling 30 tapes at T7 and T14. The statistical comparison was carried out by ANOVA and Tukey test (p<0.050) between products versus placebo and versus control. The assessments were carried out with Tewamete® 300.

Table 5 below indicates the formulations used in the tests, as well as the concentration of actives present in them:

TABLE 5

| | | Mostir. screen- | Complete Moistur. In | |
|---|---|---|---|---|
| Ref. | Formulation | ing | use | TEWL |
| 1 | Placebo | x | x | x |
| 2 | Maracok cer- | x | | x |
| | amide | | | |
| 3 | Recoverine | x | | x |
| 4 | Irwinol | x | | x |
| 5 | Filaggryl | x | x | |
| 6 | Aquaphyline | x | x | |
| 7 | Irwinol | x | | x |
| | Prodew | | | |
| | Alanine | | | |
| | Serine | | | |
| 8 | Ceramides | x | x | x |
| | Prodew | | | |
| | Alanine | | | |
| | Serine | | | |
| | Filaggryl | | | |
| 9 | Recoverine | x | | x |
| | Aquaphyline | | | |
| | Prodew | | | |
| | Alanine | | | |
| | Serine | | | |
| | Flaggryl | | | |
| 10 | Ceramides | x | x | x |
| | Aquaphyline | | | |
| | Prodew | | | |
| | Alanine/serine | | | |
| 11 | Prodew | x | x | |
| | Alanine | | | |
| | Serine | | | |
| 12 | Glycerin | x | | |

Results of the Tests:

Screening Corneometry Test:

Table 6 below shows the result of the screening corneometry test carried out:

TABLE 6

Maximum moisturizing

| | | | |
|---|---|---|---|
| Ref. Function | 2 h | 12 h | 24 h |

| | | | | |
|---|---|---|---|---|
| 1 | n/a | x | | |
| 2 | barrier | x | | |
| 3 | barrier | | x | |
| 4 | barrier | x | | |
| 5 | filagrin | x | | |
| 6 | aquaporin | x | | |
| 12 | Adsorption of water | | | x |
| 7 | Barrier, NMF | | x | |
| 8 | Barrier, NMF and filagrin | | x | |
| 9 | Barrier, NMF, filagrin and | x | | |
| | aquaporin | | | |
| 10 | Barrier, NMF, filagrin an- | x | | |
| | daquaporin | | | |
| 11 | NMF | x | | |

Remarks: The placebo is the (identical) formulation without the actives (Table 7).

On the basis of this result, one concludes that there is moisturizing synergism between the actives ceramides+Prodew/alanine/serine+Filaggryl. One notes that the test carried out for these same individualized actives results in a maximum moisturizing of 2 hours, without differing statistically from the placebo (P>0.5), whereas the mixtures thereof resulted in a maximum moisturizing of 12 hours—Formulations 7 and 8, respectively.

Complete Corneometry Test in Use:

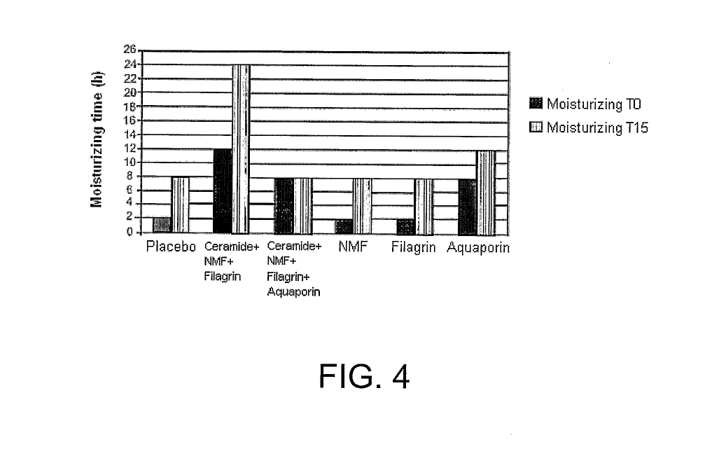

The graph that demonstrates the result of this test is illustrated in FIG. 4 of this specification.

One could notice that the formulation ceramide+NMF amino acids+filagrin provided a moisturizing for up to 12 hours with a single application (T0) even deffering from the placebo (P<0.05). This combination after 15 days of continuous use provides a moisturizing for up to 24 hours. Again, one can see that there is moisturizing synergism between the components of formulation 8.

TEWL Test:

This test was carried out for the transepidermal water loss after mechanical damages (tapes stripping) to the skin of healthful people. The smaller the TEWL, the smaller the loss of water and the better the skin barrier. In this way, one concludes that the product used reinforces the skin-barrier structure and does not permit transepitermal water losses.

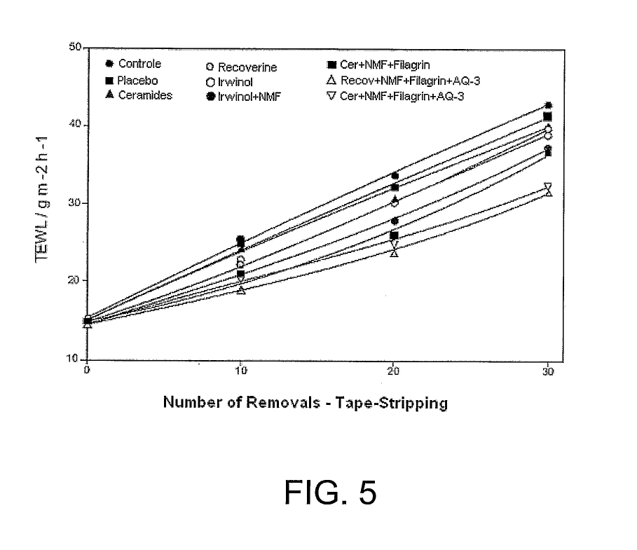

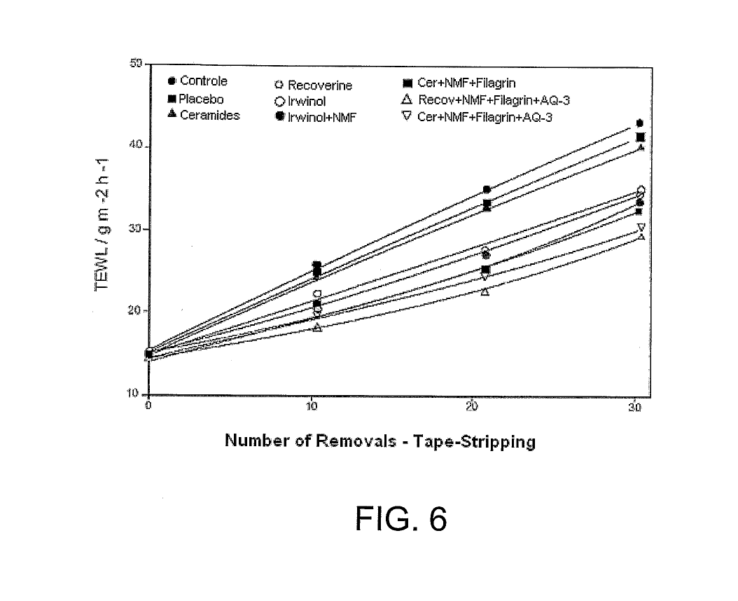

The results assessed after 7 days and fifteen days of test, respectively, are illustrated in FIGS. 5 and 6, respectively.

The control considered for this test was a group of people without use of any products in the forearm region.

One could observe that the more complex the mixture, the smaller the transepidermal water loss and, therefore, the better the reinforcement of the barrier achieved. All the formulations differ from the control region at 7 and 15 days (P<0.05).

In 7 days, all the formulas tested with the compounds individually did not present differentiation from the placebo (P>0.05). However, all the mixtures are different from the placebo (P<0.005).

In 15 days, both the formulas containing the individual compounds and the mixtures are statistically different from the placebo (p<0.05). The mixtures ceramide or Recoverine+Filagrin+Aquaporin+NMF were the ones that demonstrated smaller transepitermal water loss and, therefore, one concludes that aquaporin plays a key role ion the process of building the skin barrier, until then unknown.

Table 7 below lists the placebo formulation used in the course of the tests described above.

TABLE 7

| Component name | Concentration |
|---|---|
| XANTHAN GUM | 0.2500 |
| ALKYL ACRYLATE TR-1 | 0.6500 |
| DEMINERALIZED WATER | 87.900 |
| ISONONYL ISONONANOATE | 1.0000 |
| DICAPRILYL CARBONATE | 3.5000 |
| GLYCERYL MONOSTEARATE AND | 1.0000 |
| PEG100 STEARATE | |
| C12-15 ALKYL BENZOATE | 2.5000 |
| CETYL LACTATE | 2.0000 |
| IODOPROPINYL BUTILCARBAMATE | 0.2000 |
| PHENOXYETHANOL F | 1.0000 |

Examples of Cosmetic and/or Pharmaceutical Compositions:

The examples below are preferred variations of cosmetic and/or pharmaceutical compositions comprising a moisturizing mixture with a synergistic effect between its components, object of the present invention. In this regard, one should understand that the scope of the present invention embraces other possible variations, being limited only by the contents of the accompanying claims, which include the possible equivalents.

### Example 1

Table 8 shows a number of components and their respective concentrations, which constitute an O/W emulsion for dry sensitive skin.

TABLE 8

| Emulsion phases | Component names | Concentration (%) |
|---|---|---|
| Phase 1 | Disodium EDTA | 0.2000 |
| Phase 1 | Demineralized water | 56.5000 |
| Phsse 2 | Vegetable bidistilled | 9.0000 |
| | glycerin BXR | |
| Phase 3 | Alkyl acrylate TR-1 | 0.5000 |
| Phase 3 | Xanthan gum | 0.3000 |
| Phase 4 | Maracock ceramides | 0.2500 |
| Phase 4 | Glyceryl monostearate | 3.4000 |
| | and PEG 100 | |
| | STEARATE | |
| Phase 4 | Capric/caprylic triglic- | 3.8200 |
| | eride | |
| Phase 4 | Dcetostearylic acid | 2.2000 |
| Phase 5 | Demineralized water | 11.5000 |
| Phase 5 | Serine | 0.4800 |
| Phase 5 | Alanine | 0.4900 |
| Phase 6 | Prodew 500 | 3.5000 |
| Phase 7 | Triethanolamine | 0.6500 |
| Phsee 7 | Demineralized water | 2.0000 |
| Phase 8 | Tocoferyl (Vit. E) ace- | 0.5000 |
| | tate | |
| Phase 9 | Filaggryl LS | 2.0000 |
| Phase 10 | Phenoxyethanol F | 0.7000 |

All the percentages are calculated on the bases of the total weight (g/kg) of the composition:
Description of the process of preparing the composition described in Table 8 above: weighing and mixing the components of phase 1 and initiating the heating under mechanical stirring (75° C.); weighing and mixing the components of phase 2 under stirring; weighing and adding, under stirring, the components of phase 3 and components of phase 4 to the previously mixed components of phase 2; heating up to 75° C.; when both phases 1 and “2” are at the same temperature (75° C.), pour phase 2 into 1 under stirring for 15 minutes; initiating the cooling under mechanical stirring; preparing phase 5 and, when the temperature is close to 60° C., adding phase 5 to the previous mixture, also under stirring; adding phase 6; neutralizing the mixture with previously prepared phase 7; and when the temperature is close to 40° C., adding the components of the sequential phases (08, 09 and 10 under mechanical stirring, harmonizing after addition of each.

### Example 2

Table 9 shows a number of components and their respective concentrations, which constitute an O/W emulsion for normal sensitive skin:

TABLE 9

| Emulsion phases | Component names | Concentration (%) |
|---|---|---|
| Phase 1 | Disodium EDTA | 0.1000 |
| Phase 1 | Demineralized water | 52.3000 |
| Phase 2 | Vegetable Bidistilled | 9.0000 |
| | glycerin BXR | |
| Phase 3 | Alkyl acrylate TR-1 | 0.5000 |
| Phase 3 | Xanthan gum | 0.30000 |
| Phase 4 | Maracock ceramides | 0.2500 |
| Phase 4 | C12-15 alkyl benzoate | 3.4000 |
| Phase 4 | Cetearet-20 | 3.8200 |
| Phase 4 | Cetostearylic alcohol | 2.2000 |
| Phase 5 | Demineralized water | 11.5000 |
| Phase 5 | Serin | 0.4900 |
| | Alanine | |
| Phase 6 | Prodew 500 | 3.5000 |
| Phase 7 | Sodium hydroxide | 0.1800 |
| Phase 7 | Demineralized water | 4.0000 |
| Phase 8 | Tocopheryl acetate | 0.5000 |
| | (Vit. E) | |
| Phase 9 | Aquaphyline | 2.0000 |
| Phase 10 | Filaggryl LS | 2.0000 |
| Phase 11 | Nylon 12 | 3.0000 |
| Phase 12 | Phenoxyethanol F | 0.9600 |

All the percentages are calculated on the basis of the total weight (g/kg) of the composition:
Description of the process of preparing the composition described in Table 9 above: weighing and mixing the components of phase 1 and initiating the heating under mechanical stirring (75° C.); weighing and mixing the components of phase 2 under stirring; weighing and adding, under stirring, the components of phase 3 and components of phase 4 to the previously mixed components of phase 2; heating up to 75° C.; when both phases 1 and 2 are at the same temperature (75° C.), pour phase 2 into 1 under stirring for 15 minutes; initiating the cooling under mechanical stirring; preparing phase 5 and, when the temperature is close to 60° C., adding phase 5 to the previous mixture, also under stirring; adding phase 6; neutralizing the mixture with previously prepared phase 7; and when the temperature is close to 40° C., adding the components of the sequential phases (08, 09, 10, 11 and 12) under mechanical stirring, harmonizing after addition of each.

1. A moisturizing mixture, particularly indicated for sensitive skin, comprising:
a) at least one agent for the repair of cutaneous barrier;
b) at least one NMF-precursor amino acid; and
c) at least one stimulating agent of the synthesis of filagrin.

2. The moisturizing mixture according to claim 1, wherein said cutaneous-barrier repairing agent is present in an amount ranging from 1.0% to 49.0% by weight, on the total weight of the mixtures.

3. The moisturizing mixture according to claim 1, characterized in that said cutaneous-barrier repairing agent is an exogenous lipid and/or an agent that stimulates the synthesis of lipids of the skin barrier.

4. The moisturizing mixture according to claim 3, wherein the lipid is selected from:
i) ceramides and their analogs of space chemical structure: 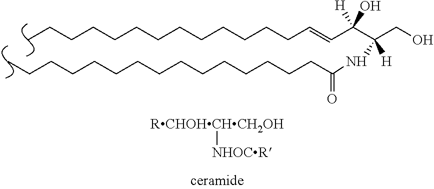
wherein R=R′ refer to C16-30:0:2;
ii) triglycerides (butters and oils) composed either individually and/or in mixture of 2 or more fatty acids selected from the group: caprylic acid (C8:0); capric acid (C10:0); lauric acid (C12:0); myristic acid (C14:0); palmitic acid (C16:0); palmitoleic acid (C16:1); margaric acid (C17:0); margaroleic acid (C17:1); stearic acid (C18.0); oleic acid (C18:10); linoleic acid (C18:2); linolenic acid (C18:3); arachidic acid (C12:0); gadoleic acid (C20:1);
iii) cyclic fatty acids with a long-chain cyclopentanic ring like chaulmougric acid, hidrocarpic acid and gorlic acid; and
iv) combination thereof.

5. The moisturizing mixture according to claim 1, wherein the NMF-precursor amino acid is present in an amount ranging from 1.0% to 98.0% by weight, based on the total weight of the mixture.

6. The moisturizing mixture according to claim 1, wherein the NMF-precursor amino acid is selected from: 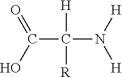
wherein R may be: H; CH₃; COH₃; C₂OH₄; C₂O₂H₃; C₃O₂H₅; C₃H₅; C₃H₇; C₄H₉; C₄NH₁₀; C₄N₂H₅; C₄N₃H₁₀; C₇H₈; C₇OH₈ and combinations thereof.

7. The moisturizing mixture according to claim 1, wherein the agent that stimulates the synthesis of filagrin is present in an amount ranging from 1.0% to 49.0% by weight, based on that total weight of the mixture.

8. The moisturizing mixture according to claim 1, further comprising an aquaporin-expression stimulating agent.

9. The moisturizing mixture according to claim 8, wherein the agent that stimulates the expression of aquaporins is selected from the polysaccharides (CH₂O)ₙ, including urocanic acids.

10. A cosmetic or pharmaceutical composition, comprising:
a) a moisturizing mixture containing at least one cutaneous-barrier repairing agent; at least one NMF-precursor amino acid and at least one filagrin-synthesis stimulating agent; and
b) a cosmetically or pharmaceutically acceptable carrier.

11. The composition according to claim 10, further comprising:
a) from 0.1% to 5.0% by weight of at least one cutaneous-barrier repairing agent;
b) from 0.1% to 10.0% by weight of at least one NMF-precursor amino acid;
c) from 0.1% to 5.0% by weight of at least one filagrin-synthesis stimulating agent;
d) from 80% to 99.7% by weight of a cosmetically acceptable carrier.

12. The composition according to claim 10 further comprising an aquaporin-expression stimulating agent.

13. Use of a moisturizing mixture comprising at least one cutaneous-barrier repairing agent, at least one NMF-precursor amino acid and at least one filagrin-synthesis stimulating agent, characterized in that it is for the preparation of a cosmetic or pharmaceutical composition indicated for moisturizing sensitive skin.

14. The use according to claim 13, characterized in that the sensitive skin exhibits atopic dermatitis and/or Ichthyosis vulgaris.

15. A cosmetic method for moisturizing sensitive skin, comprising:
a) electing a skin area to be moisturized;
b) applying to said skin area a moisturizing mixture comprising at least one cutaneous-barrier repairing agent; at least one NMF-precursor amino acid and at least one filagrin-synthesis stimulating agent.
